(19) United States
(12) Patent Application Publication
(10) Pub. No.: US 2013/0164762 A1
(43) Pub. Date: Jun. 27, 2013

(54) PROGNOSTIC TEST OF THE PROGRESSION OF A SOLID TUMOUR BY IMAGE ANALYSIS

(71) Applicants: Jean-Francois Emile, Paris (FR); Marc-Antoine Allard, Versailles (FR)

(72) Inventors: Jean-Francois Emile, Paris (FR); Marc-Antoine Allard, Versailles (FR)

(73) Assignees: Assistance Publique-Hopitaux De, Paris (FR); Universite de Versailles-ST Quentin en Yvelines, Paris (FR)

(21) Appl. No.: 13/821,675

(22) PCT Filed: Sep. 9, 2011
(86) PCT No.: PCT/EP11/65686

§ 371 Date: Mar. 8, 2013

(30) Foreign Application Priority Data
Sep. 10, 2010 (FR) 1057220

(51) Int. Cl. G01N 33/68

(52) U.S. Cl. CPC G01N 33/6893
USPC 435/7.23

(57) ABSTRACT

The present invention relates to a method for the prognosis of the progression of a solid tumour in a patient, which includes at least the following steps: a) making, from a tumour sample, a virtual slide on which a tumour front can be identified; b) quantifying, on said virtual slide made in step a), the density of cells and/or blood vessels present in a continuous area covering the tumour front and extending on either side of the tumour front over a distance of at least 0.5 mm, referred to as the quantification area; and c) deducing, from said quantification, the risks of postoperative relapse and/or the sensitivity to various antitumor treatments and/or the risks of developing metastases in said patient. Preferably, the cells to be quantified are leukocytes, such as T cells, B cells, macrophages, natural killer cells, dendritic cells, or subpopulations of these immune system cells.

Cancer is a disease characterized by abnormally high cell proliferation in normal tissue of the body, so that the survival of the body is threatened. These cells all derive from the same clone, a cancer initiator cell that has acquired certain characteristics enabling it to divide indefinitely. As the disease evolves, some cells can form a malignant tumour (a neoplasm) or spread through the body and form metastases.

Risk factors are genetics (mono- or multigenic) and environmental factors (diet, smoking, bacterial flora, etc.). A number of types of cancers appear to be increasing, for environmental or lifestyle reasons, as well as—for some cases only—because of the ageing population. The rate of cancers detected is increasing in each age segment, with improved detection also playing a role in this increase.

In 2008, 7.6 million people died of cancer, in particular in developing countries, according to a study of the International Agency for Research on Cancer (IARC, which forms part of the World Health Organization). In 2008, 56% of the 12.7 million new cases of cancer and 63% of the 7.6 million deaths associated with cancer in the world occurred in developing countries. The cancers most commonly diagnosed in the world are lung cancer (12.7%), breast cancer (10.9%) and colorectal cancer (9.7%). The highest rates of death are caused by lung cancer (18.2%), stomach cancer (9.7%) and liver cancer (9.2%). Cervical cancer and liver cancer are much more common in developing regions, while prostate cancer and colorectal cancer are more common in developed regions.

While there are many elements enabling a type of tumour to be identified with a high probability, the diagnosis of the malignance of a tumour and the prognosis for survival of a patient are today only determined based on microscope analysis (anatomopathology) of a tumour sample. This sample comes either from a biopsy (simple sample of a piece of the tumour), which can be performed, depending on the location, using different procedures (fibroscopy, skin puncture, etc.), or from a surgical specimen (tumour removed by the surgeon). The sample is usually fixed in an appropriate manner and sections are taken. These sections may be stained by traditional histochemical treatments and, if necessary, immunohistochemistry, in order to enable the identification at least of the different cell types and the limit of the tumour boundary.

The observations of the anatomical pathologist then enables the tumour sample to be classified according to internationally-recognized criteria (the TNM classification), which enable the chances of survival of the patient who has (or had) this tumour to be estimated.

The TNM classification enables patients to be grouped according to the extent of the disease in the body. It is intended i) to evaluate the prognosis, ii) to guide the indication for treatment, and iii) to compare the results of different treatment protocols. In the TNM system defined by the Union for International Cancer Control (UICC), the code T refers to the size and the local extent of the primary tumour, the code N refers to the possible involvement of lymph nodes, and the code M refers to metastases. Each letter is assigned a coefficient. The grouping of the three codes defines stages, characteristics of the probable evolution of the tumour.

The letter T symbolizes the local extent of the primary tumour. It is scored TO (when the primary lesion is not found) to T4 for the most widespread tumours. This scoring depends on the tumour volume, represented by the maximum diameter of the lesion and/or the infiltration of the neighbouring tissues and organs. The letter N, from N0 to N3, depends on the lymphatic region, more or less close to the tumour, the size of lymphadenopathies, the number of same and/any attachment to the neighbouring tissues. Finally, the letter M is scored MO in the absence of known metastases or Ml in the presence of same, regardless of their bed(s), single or multiple.

This classification was designed to give oncologists in all countries a common language to facilitate exchanges of information between physicians and researchers. It is periodically discussed and updated by specialists in the context of the UICC, which is responsible for disseminating it throughout the world in the form of an explanatory manual. It helps to codify the treatment indications. According to the tumour localizations, the combination of the three references TNM makes it possible to establish a more unified stage (from I to IV). There are, however, other classification systems, proposed by institutions or at the level of the country and its specialists, in order to improve or simplify the characterization of a cancer and the choice of treatment based thereon. For example, the anatomo-pathological classification (or pTNM) encompasses information obtained by the pathological examination of the primary tumour and the lymph nodes.

However, the TNM classification has its limits, because it is often difficult to evaluate the volume of the tumour masses by imaging (criterion T), and the amplitude of microscopic invasions. Moreover, the analysis of the tumour sample collected is only visual and requires the skills of an anatomical pathologist specializing in tumours. This analysis is therefore essentially subjective, and is time consuming.

There is therefore a need for a method for quick and objective (therefore more reliable) prognosis of the malignance of solid tumours and therefore of risks of recurrence in these patients. The prognosis method of the invention responds to this need, by providing an objective method for evaluating the prognosis of a cancer based on the analysis of a virtual slide by information processing software. The method of the invention is therefore more reliable and faster than the existing methods for evaluating the prognosis for survival of a patient with a solid tumour, in particular colorectal cancer. It moreover provides a complement to the current methods to specify the prognosis obtained by the TNM classification.

Other image analysis systems intended for prognosis have been developed in the last decade. For example, US 2004/0013292 describes the analysis of tumour sections by segmenting them in different regions. US 2003/0050553 and Rijken P. et al (Molecular Research, 1995) more specifically describe methods for determining the density of microvessels present in tumours. U.S. Pat. No. 5,616,469 deduces, therefrom, a method for prognosis of the evolution of a solid tumour in an individual.

However, all of these methods are based on the analysis of images acquired in the actual tumours, and not, as proposed by the present inventors, by comparison with the periphery thereof and on the basis of the distance from the tumour extension boundary. It has been possible to demonstrate that the infiltration of tumours by lymphocytes is of highly heterogeneous density within the same tumour in most cases, and that the maximum density peak is sometimes located outside the tumour. The inventors also demonstrated that the main factor for variability in lymphoid density was the position with respect to the tumour extension boundary (cf. example 6 of this application). For this reason, the method proposed in this application is based on the measurement of the nature and the density of the infiltration on each side of the tumour boundary.

There are numerous advantages of the method proposed in this application with respect to those known in the prior art: 1) this method integrates in its measurement the main density variation factor (i.e. the distance with respect to the tumour boundary), 2) it takes into account the peritumoural infiltration, 3) the result obtained (the classification of tumours on the basis of graphic profiles obtained) is therefore highly reproducible.

Moreover, to improve its speed of execution and its reliability, this counting technique can be fully automated owing to the use of virtual slides, image analysis software, and the standardization of measurements expressed in graphic form.

Systems for quantification of tumour-infiltrating lymphocytes (TILs) have also been developed. The lymphocytes were counted either by HE stain (Jass et al., J. Clin. Pathol. 1996), or after immunohistochemistry in situ. The counts were performed either on entire sections (Pages, N. Engl. J. Med., 2005, Laghi Lancet Oncol., 2009) or on tissue arrays (Galon et al., Science, 2006, Salama et al., J. Clin. Oncol., 2009), or manually by a pathologist (Prall. et al., Human Pathol., 2004), or with image analysis software. The software counted either a percentage of surface marked (Laghi et al., Lancet Oncol., 2009), or lymphocytes identified after segmentation of the images (Salama et al., J. Clin. Oncol., 2009). For each tumour, the studies cited used only one or two density values for the correlation with the clinical evolution.

In spite of the number of studies performed, these systems are not used in practice by physicians, because they run into problems of reproducibility due to the heterogeneous distribution of TILs in the tumours. The results of the present inventors have indeed shown that the density of TILs depends on the relative position of the tumour boundary. Thus, the densities of TILs measured at several points in the tumour are not representative of the tumour infiltration as a whole, and these measurements are unreliable. Instead, the present invention proposes measuring the distribution of TILs continuously, on each side of the tumour boundary.

### SUMMARY OF THE INVENTION

This invention relates to a method for prognosis of the evolution of a solid tumour in an individual, including at least the following steps:

a) producing a virtual slide from a tumour sample, on which a tumour boundary can be identified,

b) quantifying, on said virtual slide produced in step a), the density of cells and/or blood vessels present in a continuous area overlapping the tumour boundary and extending on each side of the tumour boundary over a distance at least equal to 0.5 mm, called the “quantification area”, preferably a rectangular area,

c) deducing from said quantification the risks of postoperative relapse and/or sensitivity to the various anti-tumour treatments and/or the risks of developing metastases in said patient.

In a specific embodiment, the rectangular quantification area is such that:

i) the perpendicular bisectors of the small side and the large side of this rectangle are respectively the normal and the tangent to the tumour boundary at said intersection point, and

ii) the intersection point of the perpendicular bisectors is a tumour boundary point.

Preferably, step a) consists at least of digitizing and recording a microscopy image of a tissue section marked by immunohistochemistry.

Preferably, the quantification step b) is performed by means of computer software and consists of sampling the cell and/or blood vessel density in a continuous set of rectangular areas of predefined width, said areas having, as their length, the width of the quantification area, and scanning the quantification area on each side of the tumour boundary, over the entire length of the quantification area.

Preferably, the result of said quantification is expressed on a graph, the analysis of which makes it possible to evaluate the risks of postoperative relapse and/or sensitivity to various anti-tumour treatments and/or the risks of developing metastases in said patient.

In a particular embodiment, said graph is such that:

i) the x-axis indicates the distance on each side of the tumour boundary,

ii) the y-axis indicates the cell and/or blood vessel density measured in each sampled area.

Preferably, the evaluation of said risks is performed:

i) by comparing the shape of said graph with a standard profile, or

ii) by calculating the area below the graph for each pre- and post-tumour boundary area, and by comparing it with threshold values,

iii) by measuring the variation slopes at two points of the graph located at a determined distance on each side of the tumour boundary and by comparing them with threshold values, or

iv) by comparing the values obtained at two points of the graph located at a determined distance on each side of the tumour boundary with threshold values.

More specifically, this invention therefore relates to a method for prognosis of the evolution of a solid tumour in an individual, including the following steps:

a) obtaining a virtual slide of a tissue section of a tumour marked by immunohistochemistry, on which a tumour boundary can be identified,

b) quantifying, on this virtual slide, the density of cells and/or blood vessels present on each side of the tumour boundary, in a continuous rectangular area extending on each side of the tumour boundary over a distance at least equal to 0.5 mm,

c) expressing these results on a graph of which the x-axis corresponds to the distance from the tumour boundary, and of which the y-axis corresponds to the quantity of cells or blood vessels measured at this distance in a rectangular surface of which the width along the x-axis is predefined, and of which the length is the width of the rectangular quantification area, and

d) performing at least one of the operations chosen from:

i) comparing the shape of said graph with a standard profile, or

ii) calculating the area below the graph for each pre- and post-tumour boundary area, and comparing it with threshold values,

iii) measuring the variation slopes at two points of the graph located at a determined distance on each side of the tumour boundary and comparing them with threshold values, or

iv) comparing the values obtained at two points of the graph located at a determined distance on each side of the tumour boundary with threshold values,

e) deducing, from step d), the risks of postoperative relapse and/or sensitivity to the various anti-tumour treatments and/or the risks of developing metastases in said individual.

In a preferred embodiment, the cells to be quantified are leukocytes such as T lymphocytes, B lymphocytes, macrophages, NK cells, dendritic cells, or sub-populations of these immune cells.

In a more preferred embodiment, the cells to be quantified are marked by immunohistochemistry and are positive for the markers CD3, CD4, CDB, CD45RO, Foxp3 and CD68.

### LEGENDS OF THE FIGURES

FIG. 1 shows, on a virtual slide showing a section of a colorectal tumour marked by immunohistochemistry with an anti-CD3 antibody so as to mark the immune cells of interest (lymphocytes), the positioning of a rectangular quantification area on each side of the tumour boundary, capable of being used in the method of the invention (A, with, at the left, the tumour area, and, at the right, the non-tumour tissue), the quantification area according to this invention, located on each side of the tumour boundary (B, the tumour boundary being identified by a black arrow), and, finally, a graph showing the automatic quantification of the density of CD3+ cells on each side of the tumour boundary in the quantification area defined in A by image analysis using the Visilog® software (C).

FIG. 2 shows, on a virtual slide showing a section of a colorectal tumour marked by immunohistochemistry with an anti-CD3 antibody so as to mark the immune cells of interest (A), the positioning of three areas of different quantifications on each side of the tumour boundary (B), and a graph showing the density of CD3+ cells on each side of the tumour boundary in the different quantification areas defined in B by image analysis using the Visilog® software.

FIG. 3 shows, on a virtual slide showing a section of a colorectal tumour marked by immunohistochemistry with an anti-CD3 antibody so as to mark the immune cells of interest (A), the positioning of three different quantification areas on each side of the tumour boundary (B), and a graph showing the density of CD3′ cells on each side of the tumour boundary in the different quantification areas defined in B by image analysis using the Visilog® software.

FIG. 4 shows different graphic profiles obtained from colorectal tumour samples. In each of the 3 graphs, a curve corresponds to 1 patient. The patients were grouped according to the profile of the curves:

A: profile 3, called “weak”, associated with a poor prognosis for survival

B: profile 2, called “strong peak”, outside the tumour, associated with a good prognosis for survival,

C: profile 1, called “strong tumour infiltration”, associated with a good prognosis for survival.

FIG. 5 shows the tumour extension boundary that can easily be detected in the great majority of cases (regardless of the stain), but that may sometimes be overtaken by tumour budding phenomena. The photographs were taken after double immunohistochemical marking, with the tumour in red (anti-cytokeratin AEl/AE3) and the lymphocytes in brown (anti-CD3).

A: On this tumour, the tumour boundary (i.e. the interface between the red and non-red areas) is easy to identify. At the highest magnification (right-hand image), many lymphocytes positive for CD3 are detected outside and inside the tumour.

B: On this other tumour, the tumour boundary is also easy to identify. At the highest magnification (right-hand image), only few lymphocytes positive for CD3 are detected.

C: On this third tumour, the tumour boundary is defined by wider compact tumour masses (delimited by a black line at the centre). At the highest magnification (rightmost image), aside from 2 smaller tumour masses, numerous isolated tumour cells are detected. On this section, there are many positive CD3 lymphocytes.

FIG. 6 shows two test curves obtained by varying the red (A) or blue (B) detection terminals for the same sample, in the same quantification rectangle (see point 5 of the experimental part). The preferred thresholds and adjustments for detection of the two colours were established on the basis of these calibration curves.

FIG. 7 shows profile type 1 called “strong tumour infiltration”: for the same colorectal tumour sample (the same patient), three different quantification areas were used (measurements 1, 2 and 3 in image A), then the presence of the lymphocyte infiltration (cells positive for CD3) in each area was quantified using the method of the invention, then represented in graphic form (B).

FIG. 8 shows profile type 4 called “weak infiltration”: for the same colorectal tumour sample (the same patient), two different quantification areas were used (measurements 1 and 2 in image A), then the presence of the lymphocyte infiltration (cells positive for CD3) in each area was quantified using the method of the invention, then represented in graphic form (B).

FIG. 9 shows profile type 2 called “strong peak”: for the same colorectal tumour sample (the same patient), throe different quantification areas were used (measurements 1, 2 and 3 in image A), then the presence of the lymphocyte infiltration (cells positive for CD3) in each area was quantified using the method of the invention, then represented in graphic form (B).

FIG. 10 shows the Kaplan-Meier survival curve of patients, between the date of diagnosis of their cancer and the detection of metastases in the lungs or the liver, as a function of their profile measured according to the method of the invention.

### DETAILED DESCRIPTION OF THE INVENTION

The prognosis method of the invention is performed ex vivo and makes it possible to evaluate the risks of postoperative relapse and/or sensitivity to the various anti-tumour treatments and/or the risks of developing metastases in a patient in whom all or some of a tumour was sampled.

It includes at least the following steps:

a) producing a virtual slide from a tumour sample, on which a tumour boundary can be identified,

b) quantifying, on said virtual slide produced in step a), the density of cells and/or blood vessels present in a continuous area overlapping the tumour boundary and extending on each side of the tumour boundary over a distance at least equal to 0.5 mm, called the “quantification area”,

c) deducing, from said quantification, the risks of postoperative relapse and/or sensitivity to the various anti-tumour treatments and/or the risks of developing metastases in said patient.

According to this invention, by “virtual slide”, is meant a digital image of a tissue sample fixed on a glass slide. In order for this virtual slide to have the same resolution that can be obtained by looking into the eyepiece of a microscope, thousands of images of the sample provided by the objective of the microscope at a sufficient magnification are acquired in series, then rearranged so as to entirely reconstruct the sample. Three steps are necessary in order to produce a virtual slide:

1) focusing: at a given position (identified by its X and Y coordinates), the sample is moved vertically (direction Z) until the scanning program decides that the image provided by the objective is as clear as possible.

2) digitization of the image: the digital image sensor acquires the photograph and stores it on the hard disk of the computer, with its X and Y information.

3) movement of the sample: the sample is moved in directions X and Y to the next position, so that the next image can be located precisely with respect to the other images.

Virtual slide acquisition techniques are now well known to a person skilled in the art. Many companies offer microscopic platforms or software capable of generating such slides using slide scanners (examples: Mirax, Aperio, Hamamatsu and Leica slide scanners) or microscopes.

These virtual slides have numerous advantages by comparison with real (“traditional”) slides holding samples: first the quality of the virtual slide cannot be altered over time. Moreover, the slides can be analyzed at any time, and remotely, since virtual slides can be shared, sent, and consulted by anyone who wishes to do so, using a computer simply equipped with image reading software. Many image reading software programs are known and routinely used in laboratories: NIH Image, Visilog, Metamorph, Histolab, etc.

The tumour sample that can be used to establish a prognosis according to the method of the invention is taken from a patient who has undergone a biopsy or a surgical operation, then treated so as to be capable of being cut into thin slices by a microtome. This treatment consists of fixing the constituents of the sample one with respect to another, so as to solidify the assembly and enable the cutting. The techniques commonly used for this treatment in laboratories are, in particular: fixation in formol (or other fixatives) or cryopreservation. After the cutting, the thin slices of tumour sample are deposited on slides, then treated by very common histology, histochemistry and/or immunohistochemistry techniques so as to show: the different tissue and cell types, the blood vessels, the tumour boundary, the cell density, etc.

Advantageously, the prognosis method according to the invention is therefore characterized in that step a) consists at least of digitizing and recording a microscopy image of a tissue section marked by immunohistochemistry.

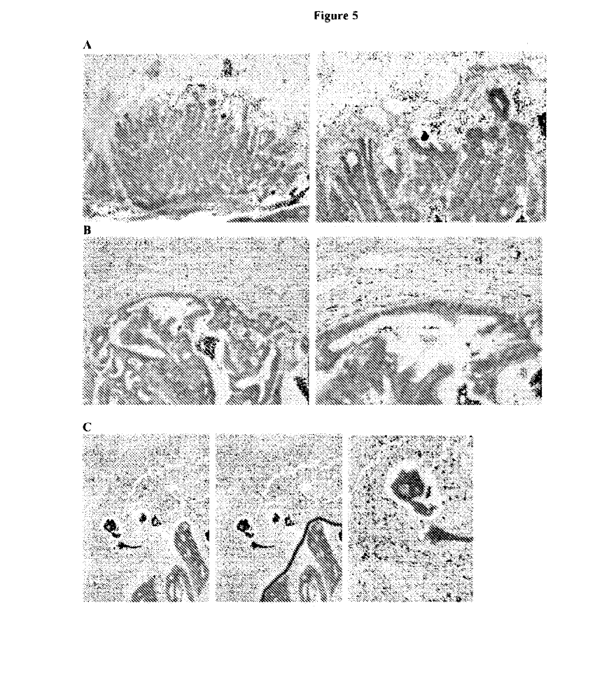

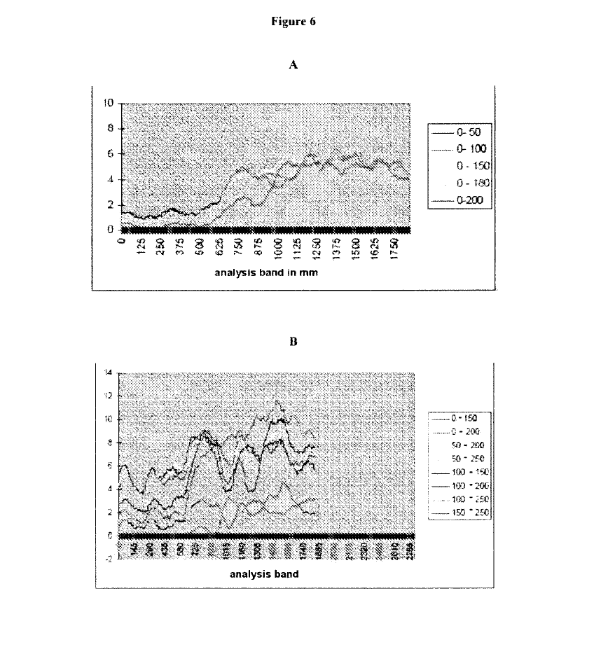

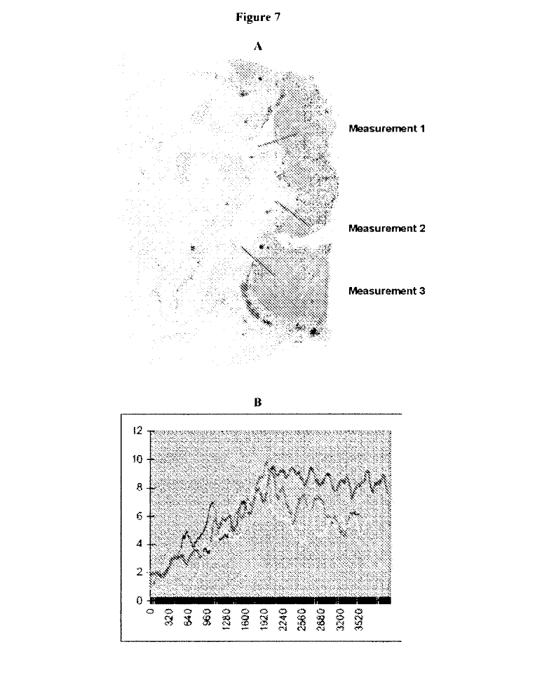

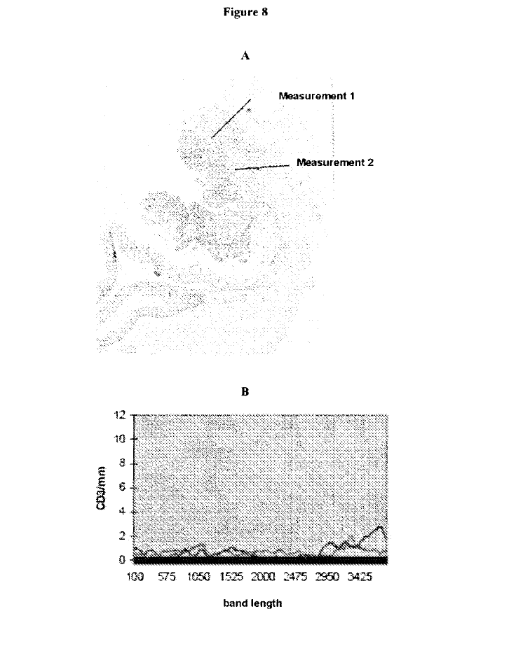

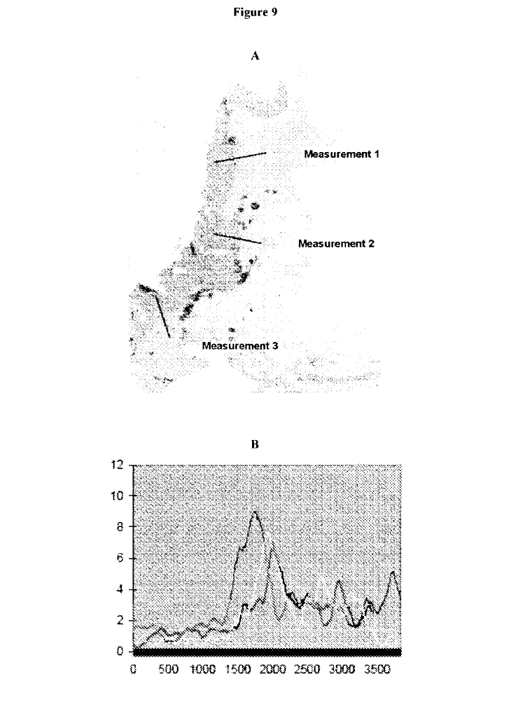

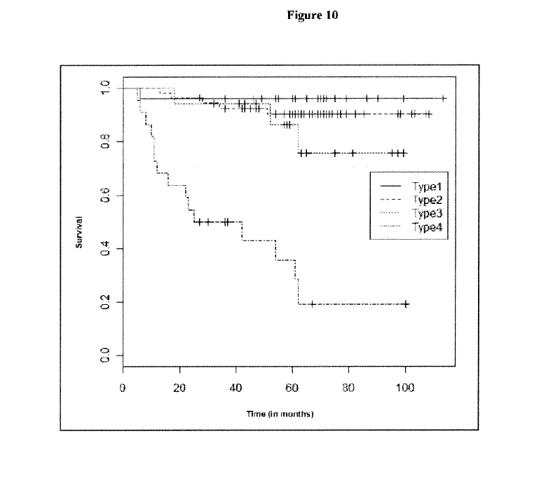

According to this invention, by “tumour boundary”, is meant the limit between the tumour tissue and the non-tumour tissue. In most tumours, and in particular in carcinomas, this limit can easily be identified with the usual histological or immunohistochemical stains. Beyond (outside) the tumour boundary, there may be, in some tumours, some isolated cells or cell masses corresponding to a tumour budding phenomenon (cf. FIG. 5C).

In addition, in the quantification area defined in the method of the invention, the tumour boundary must be likened to an arc of circle of which the centre is located inside the tumour, which corresponds to the approximation of the interface area between the tumour and the non-tumour tissues.

In other words, in the method of the invention, the quantification area is chosen specifically so that it overlaps a tumour boundary that has a curvature that is as regular as possible, i.e. a boundary having a curvature such that a tangent can be plotted in at least one point of this boundary.

The quantification area of the invention must contain the tumour boundary and must extend on either side of it over a distance at least equal to 0.3 mm, preferably 0.5 mm, and even more preferably at least equal to 0.6 mm. This area may have any shape once it is continuous (i.e. it is possible to connect any two points in the area without going outside it). Such continuous areas are, for example, circles, triangles, rhomboids, squares, rectangles, etc.

Preferably, the quantification area is rectangular, and selected as follows: first, a regular portion of the tumour boundary is identified on the virtual slide (by “regular portion of the tumour boundary”, is meant a tumour/stroma interface that can be likened to an arc of circle whose centre is located inside the tumour). Second, a point on this regular portion of the tumour boundary is defined, of which point the tangent to the tumour boundary, as well as the normal to the tumour boundary passing through this point, are plotted. Finally, the quantification is chosen so that:

i) the perpendicular bisectors of the small side and the large side of this rectangle are respectively the normal and the tangent to the tumour boundary at said intersection point, and

ii) the intersection point of the perpendicular bisectors is said point defined on the tumour boundary.

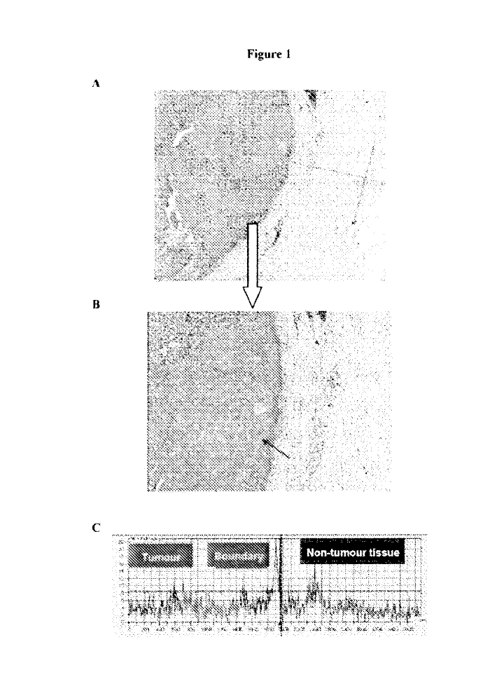

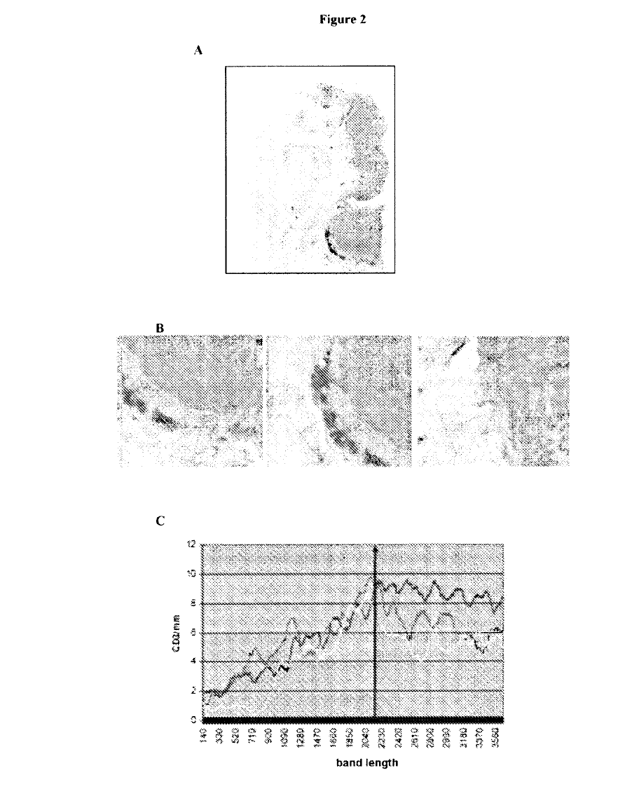

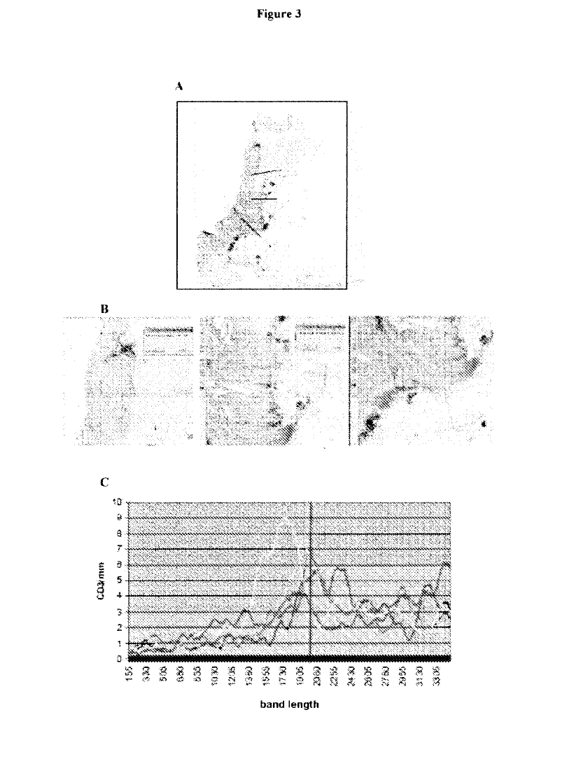

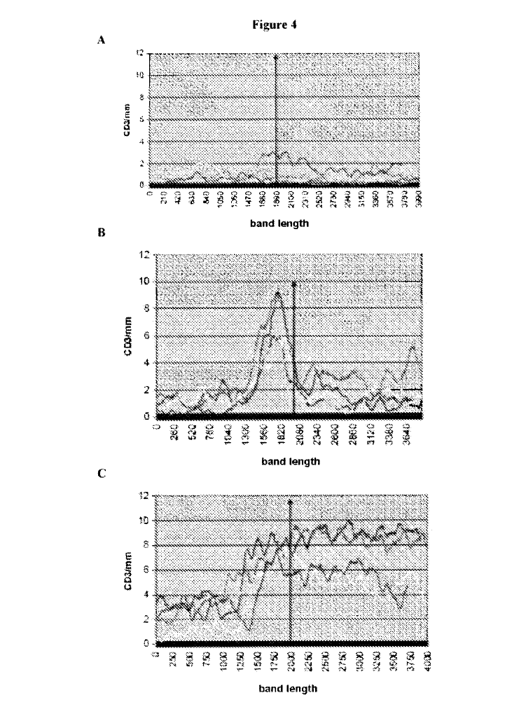

Examples of quantification areas that can be used in the method of the invention are presented in the figures of the application (cf. FIGS. 1, 2 and 3).

In the method of the invention, the perpendicular bisector of the small side must extend on each side of the tumour boundary over a distance at least equal to 0.3 mm, preferably 0.5 mm, even more preferably at least equal to 0.6 mm.

Preferably, the quantification area has a surface of 2 mm², and more preferably 3 mm². Preferably, when the quantification area is a rectangle, the length of said rectangle is at Least 0.6 mm, and the width of said rectangle is at least 0.2 mm.

The second step (step b) of the prognosis method according to the invention consists of quantifying, in the quantification area selected as described above, the density of a tissue element (vessels, collagen fibres, etc.) or cellular element (lymphocyte, macrophage, etc.) capable of being identified by its shape, its size and/or its stain, measured on each side of the tumour boundary.

The density is defined here as being the quantity of element measured, with respect to the surface unit, for example mm² or μm².

Preferably, the density of cells and/or blood vessels present in the quantification area (as defined above) is measured. Even more preferably, the density of certain cells of the immune system, present in said area or in sub-sets of said area, is measured. These cells are, for example, leukocytes, such as T lymphocytes, B lymphocytes, macrophages, NK cells, dendritic cells, or sub-populations of these immune cells.

According to the method of the invention, these cells are advantageously marked by immunohistochemistry and are positive for the markers CD3, CD4, CD8, CD45RO, FoxP3, and/or CD68. Preferably, the cells studied are positive for the CD3 (CD3⁺) marker.

The CD3 marker (or “cluster of differentiation 3”) characterizes the T lymphocytes. This well-known membrane protein complex consists of 4 different chains. In mammals, this complex is formed by a CD3γ, chain, a CD3δ chain, and two CD3ε chains. These transmembrane proteins are associated with the T cell receptor (TCR) and a ζ chain to form the “TCR complex” and generate the T lymphocyte activation signal.

The immunohistochemical marking is revealed by a peroxidase substrate (diaminobenzidine) in the examples indicated, but it is possible to use another stained substrate or another enzyme (alkaline phosphatase) or a fluorescent substrate, or even a combination of these methods.

Indeed, many studies have clearly established that the immune system is strongly involved in the control of tumours and that the density of the immune infiltration in tumours is often associated with a good prognosis for survival of surgical patients with a solid tumour (Naito Y et al., Cancer Research, 1998, Uppaluri R et al., Cancer immunity 2008, Salama P et al., Journal of clinical oncology 2009, Galon J et al., Science 2006, Badoual et al., Clinical Cancer Research, 2006).

With this respect, it should be noted that the techniques for quantification of these immune cells (T lymphocytes, regulatory T cells, etc.) in the studies conducted until now have consisted of comparing the number of immune cells present in tumour areas of variable surface (TMA (tissue-microarray) spots or fields under high microscope magnification). These areas have usually been chosen either randomly or without any clearly described methodology concerning in particular the position and the surface of the counting areas. The work of the team of J. Galon distinguished infiltrations at the boundary and inside the tumour, but does not indicate whether the samples of the boundary are located inside or, to the contrary, outside the tumour. The counts performed on complete TMA spots (generally 0.6 mm in diameter) are too broad to enable a peak to be shown, and a low variation in the position of the TMA core sample may be responsible for a high measurement variation. Although the information concerning the presence and number of immune cells in or at the periphery of tumours is recognized as being a very reliable prognostic indicator for the risk of recurrence of the tumour, no study has until now proposed the establishment of an objective and continuous profile of the number of immune cells around the tumour boundary.

An important special feature of the method of the invention is that a plurality (i.e. more than 3, and preferably more than 4) density measurements are performed, each of which is associated with a position relative to the tumour boundary.

Moreover, the method of the invention can also be very reliable by quantifying the number of blood vessels passing through the quantification area, these vessels being capable of being marked by immunohistochemistry, for example by showing the CD34 or CD31 markers (Couvelard et al., Br J Cancer 2005; Couvelard et al., Mod. Pathol., 2009).

Preferably, the solid tumour examined in order to evaluate the prognosis is a colon cancer or a rectal cancer. Colon cancer develops on the mucous membrane of the “large intestine” or colon. As colon and rectal cancers are fairly similar, they are grouped together under the term “colorectal cancer”. This is always a malignant tumour. It is the second most common cancer in women (after breast cancer) and the third most common cancer in men (after lung cancer and prostate cancer). There is a high rate of colon cancers in France: each day, 100 people learn that they have colorectal cancer. In non-smokers, this is the second leading cause of death by cancer. Men are slightly more affected than women (incidence rates of 40 and 27 percent, respectively). It is essentially a cancer that affects older people, with almost 85% of cases occurring after the age of 65 years. Its frequency appears to be increasing.

The only classification used in the pre-operative phase is the TNM classification, the 7th edition of which was published in 2010. Based on the TNM classification data, colon cancers are classified in 4 stages. The chances for recovery vary considerably from stage I to stage 1V. The treatment strategy is also adapted to each of these stages.

With regard to colorectal tumours, the density of T cells (memory T cells, regulatory T cells, and/or CD8+ T cells) has often been associated with a good prognosis (Galon J et al., Science 2006, Salama P et al., J. Clinical Oncol., 2009, Pages F et al., N. Engl. J. Med., 2005).

Preferably, the quantification provided in step b) of the prognosis method of the invention is performed using computer software. There are many computer softwares enabling the marking of virtual slides marked by immunohistochemistry to be measured. These include: NIH Image, Visilog, Metamorph, Histolab, etc. By appropriately adjusting the thresholds and parameters of the markings to be studied, the precise quantity of marked cells present in the quantification area is evaluated.

The parameterization of the image analysis software for enabling the identification then the counting of cells or tissue structures is adapted to the slide staining techniques. In the examples presented, the adjustments have been set so that they can be used without modification between a plurality of slides stained according to the same technique, and so that a slight modification of each of these adjustments causes only a slight modification in the graph obtained (cf. FIG. 6).

Advantageously, step b) of the prognosis method of the invention consists of sampling the density of cells and/or blood vessels in a continuous set of rectangular areas of predefined width, said areas having, as their length, the width of the quantification area, and scanning the quantification area on each side of the tumour boundary, over the entire length of the quantification area.

In this specific case, the computer software counts the elements to be measured (cells and/or vessels) in “slices” of the quantification area, these “slices” having a predefined width, of between 2 and 20 μm, and preferably 5 μm. This sampling step is a traditional step for image processing analyses, and the only parameter to be defined is the width of the quantification slice. The density to be quantified in step b) of the method of the invention is therefore defined here as being the quantity, per surface unit, of cells and/or blood vessels detected in these sub-sets of the quantification area.

Preferably, the result of this quantification is expressed on a graph (cf. FIGS. 1C, 2C, 3C and 4).

This graph is preferably defined as follows:

i) the x-axis indicates the distance on each side of the tumour boundary defined, which is the point of intersection of the perpendicular bisectors of the quantification rectangle,

ii) the y-axis indicates the cell and/or blood vessel density measured in each sampled area.

Preferably, the x-axis of this graph is centred on the tumour boundary point that is at the intersection of the perpendicular bisectors of the quantification rectangle, and on each side, the distance of the quantification slices from this point is indicated.

The “zero” of the x-axis can be defined:
either at one of the ends of the rectangle (in which case the increments on this axis correspond to the distance of the slices from this end point, and it is appropriate to show in the graphic where the tumour boundary point is located, with a line or an arrow, for example, cf. FIGS. 1C, 2C, 3C and 4 A, B and C),
or at the tumour boundary point (in which case the increments on this axis will be values relative to this central point).

These graphics therefore make it possible to quickly show the distribution of cells and/or vessels around the tumour boundary. Four distinct interesting and predictive profiles of survival of the patient with the tumour have been identified by the present inventors:

Profile 1: the lymphocytes are abundant inside the tumour and also outside near the boundary. The infiltration disappears as the distance from the boundary increases.

Profile 2: the immune cells are concentrated with a peak located just outside the tumour boundary. There are barely more cells inside the tumour than outside at a distance from the boundary.

Profile 3: there are few immune cells outside or inside the tumour, but a peak is distinguished outside the tumour boundary. This peak is, however, very weak.

Profile 4: the curve does not show any peak, and the average cell density is very low on each side of the tumour boundary.

Examples of such profiles are provided in FIGS. 4 A (profile 3), B (profile 2), and C (profile 1).

The present inventors were able to demonstrate that these profiles can be reproduced inside the same tumour, i.e. independently of the analysis area and the operator (cf. FIGS. 7, 8 and 9).

They also demonstrated that a more or less good prognosis is associated with these different types of graphs, and that it is therefore sufficient to establish this graph in order to improve the evaluation of the risks of postoperative relapse and/or sensitivity to various anti-tumour treatments and/or the risks of developing metastases in a patient in whom all or some of a tumour was sampled.

More specifically, the work of the present inventors made it possible to establish that the cell distribution obtained in profiles 1 and 2 is associated with a significantly lower rate of recurrence in patients who underwent surgery for a stage 11 and stage III adenocarcinoma. The immunohistochemistry with the CD3 appears to be the most effective marker. This marker is therefore preferred above all.

It is therefore possible to obtain a quick, objective and reliable prognosis of the occurrence of metastases or the survival of a patient with a tumour by means of the method of the invention by comparing the shape of said graph with a standard profile as defined above, or by calculating the area below the graph for each pre- and post-tumour boundary area, and by comparing it with threshold values, or by measuring the variation slopes at two points of the graph located at a determined distance on each side of the tumour boundary and comparing them with threshold values, or by comparing the values obtained at two points of the graph located at a determined distance on each side of the tumour boundary with threshold values.

This invention also relates to a process for image processing of a virtual slide obtained by microscopy, on which a tumour boundary is shown, including the following steps:

a) obtaining a virtual slide of a tissue section of a tumour marked by immunohistochemistry, on which a tumour boundary can be identified,

b) quantifying, on this virtual slide, the density cells and/or blood vessels located on each side of the tumour boundary, in a continuous rectangular area, and extending on each side of the tumour boundary over a distance at least equal to 0.5 mm,

c) expressing these results in a graph in which the x-axis corresponds to the distance from the tumour boundary, and in which the y-ordinate corresponds to the quantity of cells or blood vessels measured at this distance in a rectangular surface of which the width along the x-axis is predefined, and of which the length is the width of the rectangular quantification area, and

d) performing at least one of the following comparative operations:
i) comparing the shape of said graph with a standard profile, or
ii) calculating the area below the graph for each pre- and post-tumour boundary area, and comparing it with threshold values,
iii) measuring the variation slopes at two points of the graph located at a predetermined distance on each side of the tumour boundary and comparing them with threshold values, or
iv) comparing the values obtained at two points of the graph located at a determined distance on each side of the tumour boundary with threshold values,

e) estimating the risk of postoperative relapse and/or the risk of developing metastases of the patient whose tumour studied in the virtual slide was sampled.

Preferably, the quantification area mentioned in step b) of the image processing process of the invention is chosen so that:

i) the perpendicular bisectors of the small side and the large side of this rectangle are respectively the normal and the tangent to the tumour boundary at said intersection point, and

ii) the point of intersection of the perpendicular bisectors is said point defined on the tumour boundary.

The application also relates to the method for prognosis of the evolution of a solid tumour in an individual, including said steps a) through e) of the image processing process of the invention.

The standard profiles are defined as indicated above.

Preferably, it is the density of certain cells of the immune system present in said area or in sub-sets of said quantification area that is measured. These cells are, for example, leukocytes, such as T lymphocytes, B lymphocytes, macrophages, NK cells, dendritic cells, or sub-populations of these immune cells. These cells are advantageously marked by immunohistochemistry and are positive for the markers CD3, CD/1, CD8, CD45RO, FoxP3, and/or CD68. Preferably, the cells are marked with an anti-CD3 antibody and are CD3⁺.

### EXAMPLES

### 1. Cohorts

The tumours were sampled in patients with colorectal adenocarcinomas of the colon and the upper rectum who underwent surgery at the Amboise Pare hospital between January 1998 and December 2005. The criteria for inclusion were patients classified as stage II (T2 to T4, N0). The criteria for non-inclusion were the presence of lymph node involvement or distant metastasis, localization at the mid- or lower rectum, perforated tumours, history of cancer, cancers developed on polyposis or Lynch syndrome, patients lost to follow-up or deceased within the month following the intervention or having received adjuvant chemotherapy and, finally, patients in whom the resection was not microscopically complete (R1 or R2). Stage 11 was chosen because it combines patients with different prognoses and in whom the development of a discriminating prognostic factor would be of great interest.

Seventy-eight patients were included according to these criteria. All of the anatomo-pathological data (TNM classification, venous emboli, perineural sheathing, number of lymph nodes examined, budding) were recorded in a certified APIX database. The clinical data were available in the digitized FSD [Formulaire Standard des Données; Standard Data Form] clinical file.

Thirty-nine other patients with colorectal adenocarcinomas of the colon and the upper rectum, at stage III and having received, after complete surgical resection, adjuvant chemotherapy, were also analyzed.

2. Other types of Cancer Studied

Aside from the colorectal tumours mentioned above, other tumours were also studied in order to validate the benefit of the method of the invention for a wide variety of tumours. Thus, the method of the invention was tested on urothelial bladder carcinomas (n=5), pancreatic adenocarcinomas (n=5), clear cell renal carcinomas (n=5), and invasive nodular skin melanomas (n=10).

### 3. Sampling, Cutting, Treatment and Staining of the Tumour Samples

The tumour samples of the patients were preserved in paraffin in the anatomo-pathology laboratory. The slides of each patient were reviewed by an anatomical pathologist specializing in digestive oncology to verify or complete the histological data studied, and to select a paraffin block enabling the IHC to be performed. The block chosen in this last case contained the tumour sample having the largest tumour-peritumoural environment interface.

The blocks thus selected were cut with the Microm microtome into slides with a thickness of 3 μm. The slides were then preserved in an oven at 60° C. for 24 hours.

The immunohistochemistry (IHC) was performed by means of an automated Bond-Max system, Leica Microsystems, by simple marking, with the following antibodies:
anti-CD3 monoclonal antibodies (T lymphocyte markers: CD3 Pan T Cell, Dako France, dilution tq 1/50th)
anti-CD8 monoclonal antibodies (cytotoxic T lymphocyte markers: CD8 Pan T Cell, Dako France, dilution to 1/25th)
anti-CD45RO monoclonal antibodies (memory lymphocyte markers: Dakocytomation, dilution to 1/400th)
anti-FoxP3 monoclonal antibodies (regulatory T lymphocyte markers: Abcam, dilution to 1/100th).

These 4 antibodies were used in this study because they are the only antibodies mentioned in the literature for which a prognostic effect has been demonstrated.

All of the primary antibodies were revealed with the Bond Polymer Refine Detection kit (Leica Biosystems, Newcastle Ltd.) in the automated Bond-Max system (Leica).

### 4. Image Analysis

The slides were scanned using a MIRAX DESK, Zeiss, high-resolution slide scanner. The virtual slides obtained in an MRX format constituted the working support of the image analysis software (VISILOG 6.0) developed by the Noesis company (Saclay, France). An application of this software was developed specifically to measure lymphocyte infiltration. The cells of interest, marked, stained brown, are recognized in the analysis of the virtual slide. There is a rectangular surface with variable dimensions in which the marked lymphocytes will be counted.

### 5. Contrast Adjustments: Determination of the Critical Thresholds for Detection of Blue and Red

The immunohistochemistry shows the elements with the antigen of interest in brown. The counterstaining with haematoxylin appears in blue, and enables the different tissue and cell constituents to be seen better. The Visilog software separates each element of the virtual slide in a binary fashion depending on its colour: red or blue.

The intensity of red and blue is represented by the software in a numeric quantity without value, ranging from 0 (highest intensity) to 255 (lightest). It is possible to adjust the detection thresholds of each element. The red and blue threshold terminals range from 0 to 255. These adjustments make it possible to identify an element according to the intensity of its stain. Indeed, a marked lymphocyte has a cytoplasmic and/or membrane marking, and a core that will remain slightly blue due to the absence of nuclear marking. This adjustment is performed so that the blue component of the core of a marked lymphocyte is ignored and the marked lymphocyte is recognized as a red element. For FoxP3, the marking is nuclear.

### Red Detection Thresholds

If the parameters for detection of the colour red are set from 0 to 255, the software will recognize the entire range of intensities of reds. The advantage of this adjustment is that all of the red elements will be recognized, and the detection sensitivity of the lymphocytes will be 100%. The disadvantage is that this adjustment will also take into account background noise, which will lower the specificity.

The very dark red areas correspond to high-intensity immunohistochemical reactions, therefore very specific to the lymphocytes of interest. It was therefore chosen to set the lower terminal of the red detection threshold at 0, i.e. to take into account the highest intensities. Tests were conducted to determine the level of light red enabling all of the lymphocytes to be taken into account, but without background noise.

A virtual slide having a broad tumour-peritumoural area interface without separation was chosen to determine the thresholds. Five measurements were performed consecutively, with an identical analysis surface. The length of the analysis was set at 1800 μm. Each measurement differed only by the upper red detection terminal, with the lower terminal being set at 0. With a red detection terminal above 100, the curves become superimposable and the red detection threshold is no longer a critical parameter. The background noise appearing in very light red is detected by the software when the adjustment of the upper red detection terminal is over 210. The red detection terminals used are therefore 0 to 200.

For the entire study, the red detection terminals were therefore set at 0 to 200.

### Blue Detection Thresholds

Setting the blue detection terminals is also essential. Indeed, for the software, all of the elements recognized in blue are removed, i.e. no counted. Eight measurements of an identical surface were performed by varying the two blue detection terminals. The density of the lymphocyte infiltration in the area measured was very low in the peritumoural region and high in the region going from the tumour boundary to the tumour centre. By observing the curves corresponding to the different blue detection thresholds (see FIG. 6), it appeared that the curve most representative of the lymphocyte infiltrate is that obtained with the blue terminals set at 100 to 250.

All of the measurements of the study series were therefore performed with red and blue detection terminals set, respectively, at 0 to 180 and 100 to 250.

### 6. Analysis of the Lymphocyte Density in the Tumours

To evaluate the lymphocyte density, the lymphocyte density was measured for 50 tumour samples chosen randomly. For each preliminary analysis series, analysis regions of 4 mm² were chosen on each side of the tumour boundary (region 1 mm wide and 2 mm long on each side of the boundary), and the counts were performed every 5 μm. Each lymphocyte density was therefore calculated for a region of 500 μm² and was associated with its distance from the tumour boundary. The maximum and minimum values measured were 98 and 726 CD3⁺ cells per mm². The mean of the maximum density/minimum density ratio measured in the tumours was 14.2 (range of 2.5 to 55) and 31 (62%) tumours had a ratio greater than 6.

Interestingly, for 26 tumours of the group having this high ratio (i.e. for 84% of these tumours), the density variation represented a Gaussian, the peak of which was localized between 400 μm outside and 200 μm inside the boundary of invasion of the tumour.

These results make it possible to demonstrate that the lymphocyte density is highly heterogeneous in tumours and on each side of the tumour boundary, so that it is not possible to establish a reliable prognosis only on the basis of one to a few measurements of densities in intratumoural regions.

### 7. Constitution of Profiles and Groups

On each virtual slide, a plurality (i.e. more than four) of density measurements are performed, each of which is associated with the position relative to the tumour boundary. The density measurements in the examples shown were performed every 5 micrometers.

The lymphocyte infiltration from the peritumoural region to the centre of the tumour was measured according to the technique described above for the first 20 patients of the cohort.

The 20 curves obtained were classified into sub-groups according to the general profile of the curve with respect to the tumour boundary, i.e. the variations in lymphocyte densities from the periphery to the centre of the tumour. The criteria used to classify the profiles are:

1) the existence of a lymphocyte density peak,

2) its intensity,

3) its density in the peritumoural and intratumoural region.

### Profile Type 1: “Strong Intratumoural Infiltration”

The lymphocyte density is increasing. In the intratumoural region, the mean density is at least two times greater than the mean density in the peritumoural region (see FIG. 4C).

### Profile Type 2: “High Lymphocyte Density Peak at the Tumour Boundary”

This peak is defined as a lymphocyte density at a given point in the analysis band that is greater than 3 Limes the mean density of the peritumoural region. The curve shows a lymphocyte density peak at a distance of less than 300 μm from the tumour extension boundary (see FIG. 4B). This peak is said to be strong because it exceeds the density of 6 lymphocytes detected over a length of 5 μm for a width of 1000 μm. The threshold of 6 lymphocytes was defined only for curves showing lymphocytes positive for CD3 (this threshold is 4 for CD8⁺ lymphocytes, 2.5 for Foxp3+ lymphocytes and 7 for CD45RO⁺ lymphocytes).

### Profile Type 3: “Low Lymphocyte Density Peak at the Tumour Boundary”

The curve shows a lymphocyte density peak at a distance of less than 300 μm from the tumour extension boundary. This peak is said to be low because it remains below the density threshold of 6 lymphocytes detected over a length of 5 μm for a width of 1000 μm (cf. FIG. 4A).

### Profile Type 4: “Low Overall Infiltration”

The curve does not show any peak. The mean lymphocyte density is below the threshold of 2 for CD3, and CD45RO, and 1 for Foxp3 and CD8.

### 8. Homogeneity and Reproducibility of Measurements

The hypotheses that the measurements of the lymphocyte infiltration does not differ at different measurement points in the same slide was tested as follows: 2 to 4 measurements were performed on 5 consecutive slides as a function of the size of the tumour-peritumoural region interface. The curves obtained in each measurement were classified as type 1 to 4 according to the profiles described above. No difference in type of curve was observed between the measurements of the same patient, as shown in FIGS. 7B, 8B and 9B.

### 9. Results: Prognostic Impact

The curve profiles obtained for 50 patients chosen randomly were analyzed independently by 3 observers. The inter-observer reproducibility for the classification as type 1, 2, 3 or 4 was excellent with a kappa of 0.93.

In this series of 117 patients, the survival curves were represented according to the Kaplan-Meier method. The association of the potential prognosis parameters was tested by a univariate analysis, then a multivariate analysis. The usual prognostic variables wore significant in the univariate analysis, as were the density curve profiles (“lymphotum” test). However, only the density curve profiles (lymphotum test) were significantly associated with survival in the multivariate analysis.

Table 1 below shows the results obtained by these two analyses (univariate and multivariate).

TABLE 1

| | Univariate | Multivariate |
|---|---|---|
| | analysis (log | analysis (Cox |
| | scale) | model) |
| pT | 0.02 | NS |
| pN | 0.007 | NS |
| Number of lymph | 0.002 | NS |
| nodes collected | | |
| Microsatellite | 0.11 | — |
| status | | |
| Vascular embolism | 0.0004 | NS |
| Perineural | 0.007 | NS |
| invasion | | |
| Linear | <0.0001 | <0.0001 |
| quantification of | | |
| lymphocytes | | |
| (profile) | | |

The lymphotum test was also performed after marking of the tumours of these 117 patients by anti-CD8, CD45RO and FoxP3. These markers were also significantly associated with survival in the univariate analysis, then the multivariate analysis. The CD3 lymphotum test, however, was slightly better.

### BIBLIOGRAPHIC REFERENCES

Badoual et al., Clinical Cancer Research, 2006; 12: 465-72
Couvelard A et al., Br. J. Cancer, 2005; 92(1): 94-101
Couvelard A et al., Mod. Pathol., 2009; 22(2): 213-81
Galon J et al., Science, 2006; 313 (5795): 1960-4
Jass, J. Clin. Pathol. 1986; 39(6): 585-9
Jass et al., Human Pathol., 2007; 38(4): 537-545
Laghi et al., Lancet Oncol., 2009; 10(9): 877-84
Naito Y et al., Cancer Research. 1998; 58, 3491-4
Pages F et al., N. Engl. J. Med., 2005; 353(25): 2654-66
Prall et al., Human Pathol., 2004; 35(7): 808-16
Rijken. P. et al., Molecular Research. 1995; vol. 50: 141-153
Ropponen et al., J. Pathol. 1997, 182(3): 318-24
Salama P et al., Journal of Clinical Oncology, 2009, 27(2): 186-92
Uppaluri R et al., Cancer immunity, 2008; 8: 16 (journal)

1. Method for prognosing the evolution of a solid tumour in an individual, including at least the following steps:
a) producing a virtual slide from a tumour sample, on which a tumour boundary can be identified,
b) quantifying, on said virtual slide produced in step a), the density of cells and/or blood vessels present in a continuous area overlapping the tumour boundary and extending on each side of the tumour boundary over a distance at least equal to 0.5 mm, called the “quantification area”,
c) deducing from said quantification the risks of postoperative relapse and/or sensitivity to the various anti-tumour treatments and/or the risks of developing metastases in said patient.

2. Method for prognosis according to claim 1, characterized in that step a) consists at least of digitizing and recording a microscopy image of a tissue section marked by immunohistochemistry.

3. Method for prognosis according to claim 1, characterized in that the quantification step b) is performed by means of a computer software.

4. Method for prognosis according to claim 1, characterized in that said continuous area is a rectangular area.

5. Method for prognosis according to claim 4, characterized in that the rectangular area is such that:
i) the perpendicular bisectors of the small side and the large side of this rectangle are respectively the normal and the tangent to the tumour boundary at said intersection point, and
ii) the intersection point of the perpendicular bisectors is said point defined on the tumour boundary.

6. Method for prognosis according to claims 4 and 5, wherein step b) consists of sampling the cell and/or blood vessel density in a continuous set of rectangular areas of predefined width, said areas having, as their length, the width of the quantification area, and scanning the quantification area on each side of the tumour boundary, over the entire length of the quantification area.

7. Method for prognosis according to claim 1, characterized in that the result of said quantification is expressed on a graph.

8. Method for prognosis according to claim 7, characterized in that the analysis of said graph makes it possible to evaluate the risks of postoperative relapse and/or sensitivity to various anti-tumour treatments and/or the risks of developing metastases in said patient.

9. Method for prognosis according to claim 7 or 8, characterized in that said graph is such that:
i) the x-axis indicates the distance on each side of the tumour boundary defined in claim 5,
ii) the y-axis indicates the cell and/or blood vessel density measured in each sampled area.

10. Method for prognosis according to claim 9, characterized in that the evaluation of said risks is performed:
i) by comparing the shape of said graph with a standard profile, or
ii) by calculating the area below the graph for each pre- and post-tumour boundary area, and by comparing it with threshold values,
iii) by measuring the variation slopes at two points of the graph located at a determined distance on each side of the tumour boundary and by comparing them with threshold values, or
iv) by comparing the values obtained at two points of the graph located at a determined distance on each side of the tumour boundary with threshold values.

11. Method for prognosing the evolution of a solid tumour in an individual, including the following steps:
a) obtaining a virtual slide of a tissue section of a tumour marked by immunohistochemistry, on which a tumour boundary can be identified,
b) quantifying, on this virtual slide, the density of cells and/or blood vessels present on each side of the tumour boundary, in a continuous rectangular area as defined in claim 5, and extending on each side of the tumour boundary over a distance at least equal to 0.5 mm,
c) expressing these results on a graph of which the x-axis corresponds to the distance from the tumour boundary, and of which the y-axis corresponds to the quantity of cells or blood vessels measured at this distance in a rectangular surface of which the width along the x-axis is predefined, and of which the length is the width of the rectangular quantification area, and
d) performing at least one of the operations as defined in claim 10:
e) deducing, from step d), the risks of postoperative relapse and/or sensitivity to the various anti-tumour treatments and/or the risks of developing metastases in said individual.

12. Method for prognosis according to any one of the previous claims, characterized in that the cells to be quantified are leukocytes such as T lymphocytes, B lymphocytes, macrophages, NK cells, dendritic cells, or sub-populations of these immune cells.

13. Method for prognosis according to any one of the previous claims, characterized in that the cells to be quantified are marked by immunohistochemistry and are positive for the markers CD3, CD4, CD8, CD45RO, FoxP3 and CD68.
